(19) United States
(12) Patent Application Publication
(10) Pub. No.: US 2025/0235604 A1
(43) Pub. Date: Jul. 24, 2025

(54) DEVICES AND METHODS FOR MEDICAL AND/OR SURGICAL IRRIGATION

(71) Applicant: Jeffrey R. MARCUS, Chapel Hill, NC (US)

(72) Inventor: Jeffrey R. MARCUS, Chapel Hill, NC (US)

(21) Appl. No.: 18/718,360

(22) PCT Filed: Dec. 7, 2022
(86) PCT No.: PCT/US2022/052124

§ 371 Date: Jun. 10, 2024

Related U.S. Application Data
(60) Provisional application No. 63/313,402, filed on Feb. 24, 2022.
(60) Provisional application No. 63/288,276, filed on Dec. 10, 2021.

(51) Int. Cl. A61M 3/02; A61J 1/14

(52) U.S. Cl. CPC A61M 3/0279; A61M 3/0262; A61J 1/1475

(57) ABSTRACT

A system for irrigation and debridement of a wound site includes an irrigation vessel, the irrigation vessel having a at least one opening and containing an irrigant solution; and a deployment head configured to couple to the at least one opening, the deployment head is configured to facilitate egress of the irrigant solution from within the irrigation vessel; wherein the deployment head, comprises: a base, the base configured to couple to the at least one opening; a stem coupled to the base at a first end, the stem having an inner channel that is in fluid communication with the irrigation vessel and configured to allow flow of the irrigant solution therethrough; and an outlet coupled to the base at a second end, the outlet having at least one aperture disposed therein, wherein the at least one aperture is fluidly coupled to the inner channel, the at least one aperture configured to expel the irrigant solution from the vessel; wherein the outlet comprises a working surface configured to debride a wound site.

### CROSS-REFERENCE TO RELATED PATENT APPLICATIONS

This applications claims priority to U.S. Provisional Patent Application No. 63/288,276, filed Dec. 10, 2021, and U.S. Provisional Patent Application No. Application 63/313,402, filed Feb. 24, 2022, each of which is incorporated herein by reference in their entirety.

### FIELD

The present technology is generally related to cleansing and decontamination of wounds and anatomic sites.

### BACKGROUND

Surgical sites can potentially leave patients vulnerable to infections at the surgical site. Accordingly, methods are systems are required to reduce likelihood of infection by removing, diluting, or reducing contaminants at the surgical site.

### SUMMARY

One aspect of the present disclosure relates to a system including an irrigation vessel, the irrigation vessel having at least one opening and containing an irrigant solution. The system further includes a deployment head configured to couple to the at least one opening, where the deployment head is configured to facilitate egress of the irrigant solution from within the irrigation vessel. The deployment head includes a base, where the base is configured to couple to the at least one opening. The deployment head also includes a stem coupled to the base at a first end, where the stem has an inner channel that is in fluid communication with the irrigation vessel and is configured to allow flow of the irrigant solution therethrough. The deployment head also includes an outlet coupled to the base at a second end, where the outlet has at least one aperture disposed therein, where the at least one aperture is fluidly coupled to the inner channel, and where the at least one aperture is configured to expel the irrigant solution from the vessel. The outlet includes a working surface configured to debride a wound site.

In various embodiments, the working surface is defined by an outer surface of the outlet, where the outer surface includes a plurality of protruding members extending from the outer surface, and where each of the protruding members is configured to mechanically debride at least a portion of the wound site. In some embodiments, the outlet is bulbous, where the plurality of protruding members is disposed about a circumference of the outlet. In other embodiments, the plurality of protruding members is arranged in substantially linear sections. In yet other embodiments, the plurality of protruding members includes ridges. In various embodiments, the plurality of protruding members include furrows. In some embodiments, the plurality of protruding members include spikes. In other embodiments, the at least one opening of the irrigation vessel includes a first opening and a second opening. In yet other embodiments, the system also includes a second container, where the second container is configured to contain a fluid, where the second container is further configured to couple to at least one of the first opening or the second opening. In various embodiments, the deployment head is configured to couple to the first opening and the second container is configured to couple to the second opening. In some embodiments, the irrigation vessel is configured to be removably coupled to the deployment head. In other embodiments, the irrigant solution includes chlorhexidine gluconate (CHG), saline, a mixture thereof, or other irrigation solution. In yet other embodiments, the at least one opening of the irrigation vessel includes at least one seal. In various embodiments, the at least one seal is a hermetic seal.

Another aspect of the present disclosure relates to a method of irrigating and debriding a wound site. The method includes filling a vessel with an irrigant solution, where the vessel includes at least one opening. The method further includes coupling a deployment head to the at least one opening of the vessel and applying a force to the vessel, where applying the force to the vessel causes egress of the irrigant solution from the vessel through the deployment head. The deployment head includes a stem fluidly coupled to the base, where the stem includes an inner channel that is in fluid communication with the irrigation vessel. The deployment head further includes an outlet coupled to the base, where the outlet has at least one aperture disposed therein, and where the at least one aperture is fluidly coupled to the inner channel. The outlet includes a working surface configured to debride a wound site.

In various implementations, filling the vessel includes coupling a second container to the at least one opening. In various implementations, coupling the second container to the at least one opening includes inserting a trocar through a seal of the at least one opening. In some implementations, the method further includes adjusting an angle of the stem. In other implementations, the method also includes debriding the wound site by mechanically loosening at least one of tissue or debris from the wound site via one or more protrusions disposed on the outlet. In yet other implementations, the deployment head further includes tubing extending between the base and the stem.

This summary is illustrative only and should not be regarded as limiting.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a side cross-sectional view of an irrigant vessel assembly, according to at least one embodiment.

FIG. 2A is a side cross-sectional view of a vessel of the irrigant vessel assembly near an opening of the vessel, illustrating an outlet connector coupled to the vessel, according to at least one embodiment.

FIG. 2B is a side cross-sectional view of the vessel of the irrigant vessel assembly near an opening of the vessel, illustrating a trocar interfacing with the opening of the vessel, according to at least one embodiment.

FIG. 3A is a side cross-sectional view of the vessel of the irrigant vessel assembly near an opening of the vessel, illustrating a trocar interfacing with a threaded opening of the vessel, according to at least one embodiment.

FIG. 3B is a side cross-sectional view of the vessel of the irrigant vessel assembly near an opening of the vessel, illustrating an outlet connector coupled to the opening of the vessel assembly, according to at least one embodiment.

FIG. 3C is a side perspective view of the vessel of the irrigant vessel assembly near an opening of the vessel, illustrating a seal disposed at the opening of the vessel, according to at least one embodiment.

FIG. 4 is a cross-sectional view of an irrigant vessel assembly, according to at least one embodiment.

FIG. 5 is a cross-sectional view of an outlet connector of the irrigant vessel assembly of FIG. 4, according to at least one embodiment.

FIG. 6A is a side view of a tip portion of the outlet connector of FIG. 5, according to at least one embodiment.

FIG. 6B is a cross-sectional view of the tip portion of FIG. 6A taken along line 6-6 of FIG. 6A, according to at least one embodiment.

FIG. 6C is a perspective view of the outlet connector of FIG. 5 near the tip portion, according to at least one embodiment.

FIG. 6D is a perspective view of the outlet connector of FIG. 5 near the tip portion, according to at least one embodiment.

FIG. 6E is a perspective view of the outlet connector of FIG. 5 near the tip portion, according to at least one embodiment.

FIG. 6F is a side view of a spout portion of the outlet connector of FIG. 5, according to at least one embodiment.

FIG. 6G is a side view of the spout portion of the outlet connector of FIG. 5, according to at least one embodiment.

FIG. 6H is a side perspective view of an overlay for the tip portion of the outlet connector of FIG. 5, according to at least one embodiment.

FIG. 6I is a side perspective view of an overlay for the tip portion of the outlet connector of FIG. 5, according to at least one embodiment.

FIG. 6J is an end view of the tip portion of the outlet connector of FIG. 5, according to at least one embodiment.

FIG. 6K is an end view of the tip portion of the outlet connector of FIG. 5, according to at least one embodiment.

FIG. 6L is an end view of the tip portion of the outlet connector of FIG. 5, according to at least one embodiment.

FIG. 7A is a side view of a vessel within an irrigant vessel assembly, according to at least one embodiment.

FIG. 7B is a side view of the vessel of FIG. 7A near an opening of the vessel, illustrating engagement of a trocar tip with the opening, according to at least one embodiment.

FIG. 8 is a side view of an irrigation vessel, according to at least one embodiment.

FIG. 9 is a side view of a decanter configured to engage with the irrigation vessel of FIG. 8, according to at least one embodiment.

FIG. 10 is a side view of a cap configured to engage with the irrigation vessel of FIG. 8, according to at least one embodiment.

FIG. 11 is a side view of a cap configured to engage with the irrigation vessel of FIG. 8, according to at least one embodiment.

FIG. 12 is a side view of a cap configured to engage with the irrigation vessel of FIG. 8, according to at least one embodiment.

FIG. 13 is a side view of an irrigation vessel, according to at least one embodiment.

FIG. 14 is a side view of an irrigation vessel, according to at least one embodiment.

FIG. 15A is a side perspective view of a transfer component configured to engage with an irrigation vessel, according to at least one embodiment.

FIG. 15B is a side perspective view of a connector within the transfer component of FIG. 15A, according to at least one embodiment.

FIG. 16 is a side perspective view of an irrigation vessel, according to at least one embodiment.

FIG. 17 is a side perspective view of an irrigation vessel, according to at least one embodiment.

FIG. 18 is a side view of an irrigation assembly, according to at least one embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Wounds and surgical incisions are two types of disruption to protective surface barriers of the body. The difference between a wound and an incision is that the term “wound” typically connotes the consequence of trauma or accident, and incisions are surgically performed carefully and for a particular purpose. In either event, the mechanical protective function of our surface defense (skin, mucous membranes, etc.) is breached, and a risk of infection emerges by virtue of the introduction of potential infectious agents to tissues unadapted to coexist with them.

There are similarities and differences with regard to risk for infection consequent to surgical incisions versus acute traumatic wounds. In surgery, steps are taken to decolonize and decontaminate prior to initiation of skin/mucosal incision. Accordingly, introduction of pathogens is less likely to result from the incision process itself. A number of factors have been implicated for increased risk of surgical site infection. Some factors include intraoperative factors such as prolonged operation time, which can permit increased exposure to pathogens present even in an operating room environment. For acute traumatic wounds, the event itself directly introduces contaminants to open bodily tissues. High-energy trauma is caused by major force (e.g., ballistic, crush, blast, motor vehicle collision, falls, etc.) in which a high amount of kinetic energy is transferred to the body resulting in tissue damage. Under these circumstances, contamination by pathogens frequently is concurrent and makes such injuries highly susceptible to infection. For example, lawnmower and chainsaw injuries devitalize tissues sharply, erratically, and extensively while impacting organic contaminants in the tissues. Similarly, ballistic and blast injuries impose thermal and kinetic energy while also introducing environmental pathogens into compromised tissues.

In traumatic injuries of the lower extremity, as an example, extended time from injury to definitive reconstruction is known to decrease the rate of limb salvage. However, the window of opportunity for successful salvage can be reliably extended by implementation of various clinical measures, including adequate surgical debridement and irrigation. According to Valerio et al., “Soft tissue injuries must undergo early, aggressive, and serial debridement as well as irrigation of all nonviable skin, muscle, and other soft tissues . . . . These measures rid the wound of nonviable tissues, which are a nidus for infection, while subsequently diluting any contaminants to the wound bed . . . ” McCulloch I and Valerio I. Lower Extremity Reconstruction for Limb Salvage and Functional Restoration—the Combat Experience. Clin. Plastic Surg. 48 (2021) 349-361. Furthermore, use of active antimicrobial agents (e.g., antiseptics and antibiotics) as additives to surgical irrigation fluids has become common practice to reduce bacterial burden and incidence of further infection. When infection/contamination occurs in combination with tissue devitalization, the combination of irrigation and mechanical debridement are consistent needs.

The use of irrigation in surgical procedures outside the setting of trauma is also quite prevalent with approximately 30-50% of general, orthopedic, plastic, neuro, and gynecologic surgeons routinely employing antibiotic irrigation in the course of surgery. However, research is lacking in support of the use of anti-microbial agents in irrigation to reduce the incidence of surgical site infections (SSI's) in elective surgical procedures.

Until recently, the ubiquitous antimicrobial surgical irrigant was bacitracin. Bacitracin is an antibiotic originally introduced in 1948 in an injectable form and was only approved by the FDA for use in treating pediatric empyema with gram-positive organisms. Bacitracin has reached widespread use for surgical irrigation. On Jan. 31, 2020, the United States Food and Drug Administration issued a voluntary recall of bacitracin for injection due to efficacy and safety concerns, including anaphylactic reactions. Given its pervasive use throughout the United States, the shift has catalyzed a critical reevaluation of clinical practice of surgical irrigation and reassessment of existing agents and the evidence to support their use.

Surgical irrigants are considered topical agents, which historically have not been managed centrally by a pharmacy, but rather made accessible at the point of care. At present, there are a number of commercially available antiseptic solutions available in a compounded form, including povidine iodine, hydrogen peroxide, chlorhexidine gluconate (CHG), acetic acid, sodium hypochlorite, and hypochlorous acid.

The germ theory of disease, which evolved following the work of Pasteur and Koch in the mid- to late-1800's, provides the modern basis for understanding the pathogenesis of infection. Microorganisms living throughout the environment and even on or within a living host, can invade, reproduce, and cause disease. The human body is equipped with a variety of defense mechanisms, which allow it thrive readily in an environment that abounds with risks visible only microscopically. Among the defenses is the mechanical barrier function of our skin and mucous membranes.

As described above, a wound is an injury to the living tissue, such as the skin or mucosa, caused by an impact (e.g., sharp or otherwise) that results in breach of the barrier function of the tissues. Accordingly, a “wound” can convey a wide range of injuries from abrasion, to puncture, laceration, surgical incision, burn, blast, or degloving. Once the mechanical protective function of the bodily surface defense has been breached, risk of infection emerges through introduction of potential infectious agents to tissues unadapted to coexist with them.

Many factors increase risk of infection following wounding. Systemic factors associated with certain health conditions play a major role for those affected by such states. Conditions of immune-compromise, such as diabetes or immune deficiency, increase infection risk compared to an otherwise equivalent injury without said conditions. Wounds occurring in areas affected by hypoperfusion (e.g., radiated tissues, ischemia secondary to peripheral vascular disease, etc.) are also at a greater relative risk.

Risk factors for wound infection related to the physical injury itself, apart from any systemic predispositions, are multifactorial. Two factors that contribute to infection development include loss of barrier function and the presence of contamination. A third contributing risk factor is the presence of devitalized tissues at the site of injury. The size of an injury may not be as important as the presence and concentration of microbial contamination. For example, the incidence of infection is generally very low in an elective surgical procedure requiring extensive incision and exposure as exposure to pathogens in this setting is limited by preparation and technique. Conversely, the likelihood of infection following a puncture wound from a cat bite is substantial if not properly and expeditiously treated. Generally, the first step in managing a laceration is to hold it under running water as irrigation mechanically removes surface contaminants and dilutes those left behind. Risk may be further mitigated by irrigation in combination with systemic antimicrobial agents for high-risk situations. Accordingly, when a barrier breach permits infectious microbes in high concentration or of a highly infectious nature, appropriate treatment (irrigation and anti-microbial delivery) and the timing can reduce the risk of infection.

The utility of irrigation in prevention of infection is further illustrated in the setting of abrasion. A superficial (i.e., partial thickness) loss of skin also reduces barrier function and can lead to infection, ranging from cellulitis to more aggressive and invasive soft tissue conditions. Early and frequent cleansing of the injury with an irrigant solution is associated with more rapid epithelialization and reduced incidence of infection. If treatment is delayed or inadequate, such wounds can develop a surface fibrinous exudate that, once desiccated, facilitates bacterial reproduction. Thus, the importance of expedient treatment is exemplified.

The conflicts in Iraq and Afghanistan represent the longest sustained engagement in American military history. McCulloch I and Valerio I. Lower Extremity Reconstruction for Limb Salvage and Functional Restoration—the Combat Experience. Clin. Plastic Surg. 48 (2021) 349-361. Numerous analyses of injuries incurred by American combat casualties demonstrated a predominance of survivable high-energy injuries to the extremities and maxillofacial regions. These observations are explained by the widespread use of protective body armor, which leave the extremities and facial regions relatively unprotected, while reducing the incidence of life-threatening thoracic and head injury. The use of improvised explosive devices has further introduced the potential for high-energy injury designed to create debilitating composite tissue wounds.

In the context of lower extremity salvage associated with high-energy injury and tissue loss, a landmark 1986 publication by reconstructive surgeon Marko Godina demonstrated a higher rate of limb salvage and reduced infection among 500 patients when reconstructive surgery was performed within 72 hours of injury. Later, surgeons further determined that the window of opportunity for successful salvage could be reliably extended with adequate surgical debridement, including irrigation.

Timing for wound treatment was further brought to attention over the course of the Middle East conflicts. In the height of the conflict, the mandate by then Secretary of Defense Robert Gates that all military medical evacuation occur in less than 60 minutes to surgical facilities directly contributed to the lowest mortality of any conflict in history. With the later reduction of forces deployed to more countries and in remote zones, the paradigm could not be maintained. At present, trained medics must work among smaller teams without relative oversight for longer periods before extrication and transfer are feasible (Keenan). In the setting of high-energy injuries, such as those to the extremities or head and neck, the term “prolonged field care” refers to the longer period of time (i.e., days versus hours) during which personnel must manage clinical situations with the intent to limit mortality and morbidity. Focused and specific training, preparation, and supplies are required to provide prolonged field care. A wound cannot be left without care for 72 hours and be expected to fare satisfactorily. Wound care ideally includes debridement and serial irrigation. The materials and devices to perform such care should be available to do so effectively.

In civilian care, and in the context of tertiary trauma referral centers, the concept of wound care is equally relevant. Delays in care for open wounds (e.g., those to the extremities or the face) still occur for a variety of reasons. The skills to manage high-energy injuries may not be present among personnel at an initial arrival facility. However, even when medical professionals with expertise are not available, basic care, including serial irrigation, as prescribed by prolonged field care concepts could be provided quickly by those with basic skills until transfer.

One aspect of the present disclosure relates to devices and methods for deployment of medical/surgical irrigant. Such devices may be suitable for medical and/or surgical irrigation in a wide range of clinical care environments including, but not limited to, open wound and intra-cavitary treatment. An outlet connector (“delivery head”) may couple to an irrigant vessel. The delivery head may be structured to deploy an irrigant, accommodate access ports used to fill the vessel from a secondary source of irrigant, and support a closed (i.e., sterile) environment during the process of filling to deployment. In at least one embodiment, the delivery head may couple to a vessel at the same access site used for transfer of sterile solution to the vessel. In at least one embodiment, the delivery head includes an elongated stem or spout of desired length, inner/outer diameter, and angulation through which the irrigant is deployed. In some embodiments, the stem may also include a terminal end with one or more features that facilitate its use as a tool to access anatomic recesses and/or physically debride tissues in the course of irrigation.

In at least one embodiment, the deployment head may include a nozzle, jet, and/or sprayer. In some embodiments, the deployment head may be coupled to a receiving port on an irrigant vessel, where the irrigant vessel contains irrigant solution for deployment (with pressure if desired) to tissues. In other embodiments, the vessel, including the deployment head, may be structured to access tissue and anatomic recesses, cavities, and wounds for irrigation while also providing a combination of tissue protection and mechanical debridement.

In various embodiments, the irrigant vessel is a container or reservoir for a fluid, such as an irrigating solution, for delivery to a surgical site, wound, etc. In some embodiments, the container may be filled with a pre-prepared or compounded solution or diluent. The solution or diluent may be prepared using a closed, sterile method and protocol. The container may have at least one opening or port with a lid, cap, or other attachable and/or removable component to facilitate selective access to the container. In some embodiments, the deployment head may interface with the port through the same mechanism as the removable access (e.g., threads, spike, friction fit, etc.), or through another mechanism. In at least one embodiment, the deployment head itself is a cap or lid, which may be coupled to the irrigation vessel via one or more threads. In other embodiments, other coupling methods may additionally or alternatively be employed including, but not limited to, friction fit.

In some embodiments, the vessel may be filled from a secondary source (e.g., through a port which is open or sealed with a cap, lid, or hermetic seal) permitting filling access but not configured to couple to a deployment head. In such embodiments, the vessel may include more than one port or opening such that a port separate from the port used for filling from the secondary source may be used to couple to a deployment head. In other embodiments, the irrigant vessel may include an integrally formed deployment head, where the integrally formed deployment head is separate from the port. In at least one embodiment, the irrigant vessel includes a port that is structured include an interface that facilitates access to the port to fill the vessel from a secondary source and permits attachment of the deployment head.

In at least one embodiment, the irrigant vessel may be accessed and filled via a port having a membrane/seal (e.g., at its base, at its terminus, etc.). For example, in some embodiments, the port of the irrigant vessel may be similar to an access site on an intravenous fluid bag. In other embodiments, the port can be accessed using a trocar (i.e., spike) connector. In another embodiment, the access port may be a threaded aperture configured to engage with a corresponding threaded attachment. In various embodiments, the same interface (i.e., the port) used for access and filling may be used to attach a deployment head. In some embodiments, the irrigant vessel may be fabricated with the deployment head already attached, and the access port for filling may be separately disposed from the deployment head).

In some embodiments, an irrigant delivery system includes a delivery mechanism for the irrigant vessel, where the delivery mechanism may be configured to couple to the vessel such that the irrigant solution remains substantially closed to the environment during solution compounding, transfer to the vessel, and delivery to the patient. In various embodiments, the delivery mechanism may couple to the irrigation vessel regardless of whether the vessel was filled using a closed transfer technique or an open technique (e.g., open decanting). In some embodiments, the irrigant solution (i.e., the irrigant solution contained in the irrigant vessel) contain an active agent, such as an antiseptic or antibiotic. In various embodiments, the deployment head may be configured to provide both irrigation and debridement in a wide range of medical and surgical settings.

The term “deliver” refers to the process in which solution from the irrigant vessel is actively or passively ejected onto a subject, which may include, but is not limited to, a patient, wound, anatomic cavity, graft, implant, device, etc. Delivery, deployment, disbursement, emission, application, and ejection are all among terms that may be used to describe functionality provided by the subject invention. “Active” ejection refers to a process in which an irrigation vessel/reservoir is pressurized for delivering an irrigant at a higher velocity, flow, etc., such as by manual manipulation (e.g., squeezing) or with the aid of a machine. “Passive” refers to any other method that is not “active.” For example, an irrigation solution may be delivered via fluid force, such as by hanging an irrigating vessel or other container such that gravity forces the fluid generally downward towards the terminus of a deployment head.

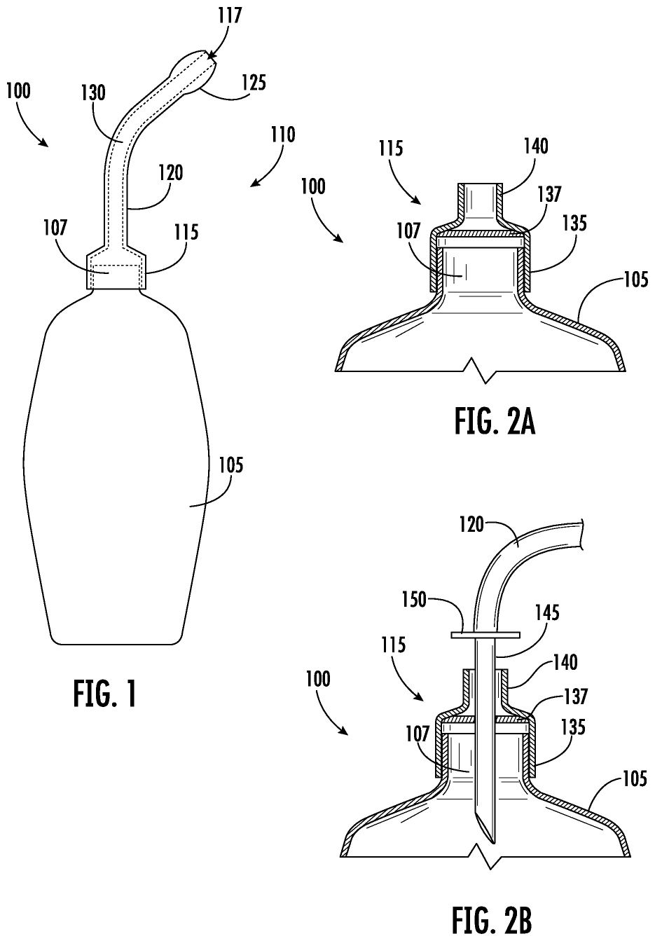

FIG. 1 depicts an irrigation system 100, which includes an irrigation vessel 105 according to at least one embodiment. The vessel 105 includes at least one opening 107 (e.g., receiving port), where a deployment head (“outlet connector”) 110 is coupled to the opening 107 via a base 115 (“interface port”) disposed at a first end of the deployment head 110. In some embodiments, the vessel 105 may have a diameter or width of approximately 2.5 inches and a height of approximately 7 inches. In various embodiments, the deployment head 110 may be configured such that the base 115 is approximately 1 inch in height, the stem is approximately 3.25 inches in length. In some embodiments, the deployment head 110 may have a total length of approximately 6 inches. In other embodiments, the stem may be approximately 5.25 inches in length. In various embodiments, the base 115 is coupled to the opening 107 via one or more threads, a friction fit, a bayonet connector, a needle, and/or a non-needle connector. In some embodiments, the deployment head 110 is freely rotatable relative to the opening 107. In some embodiments, the deployment head 110 is fixed relative to the opening 107 (e.g., by tightened threads). In various embodiments, the connection between the opening 107 of the vessel 105 and the deployment head 110 may be permanent (e.g., by deformation of at least one of the base 115 or parts of the opening 107, or a piercing of a seal) or temporary (e.g., by reusable threads disposed at the opening 107 and the base 115).

In various embodiments, at least one of the vessel 105 or the deployment head 110 may be made of one or more sterilizable (e.g., via an autoclave, chemical treatment, etc.). In some embodiments, the vessel 105 and/or the deployment head 110 may include or be made of one or more metallic materials. In other embodiments, the vessel 105 and/or the deployment head 110 may include or be made of one or more non-metallic materials (e.g., one or more biocompatible plastics). In various embodiments, the vessel 105 may be deformable to facilitate pressurization by a user (e.g., squeezing) to expel irrigation solution from the vessel 105 (i.e., via the deployment head 110). In some embodiments, the vessel 105 may be generally cylindrical in shape. In other embodiments, the vessel 105 may be rectangular. In yet other embodiments, the vessel 105 may have any suitable shape configured for storing irrigation solution therein. As shown in FIG. 1, the vessel 105 may have a greater width within a midsection of the vessel 105 as compared to a width adjacent a top (i.e., near the opening 107) or bottom of the vessel 105. In various embodiments, the entire vessel 105 may be at least somewhat deformable. In other embodiments, the vessel 105 may include one or more deformable regions, where the vessel 105 may be pressurized responsive to deformation within the one or more deformable regions while remaining substantially rigid outside of the one or more deformable regions. Although FIG. 1 shows the opening 107 disposed within a top portion of the vessel 105, in other embodiments, the opening 107 may be disposed anywhere within the vessel 105 (e.g., bottom, side, etc.).

As shown in FIG. 1, the deployment head 110 includes an outlet 125 disposed opposite the base 115, where the outlet 125 is connected to the base 115 via a stem 120. The outlet 125 is disposed at a distal, terminal end (i.e., opposite the base 115) of the deployment head 110 and includes at least one opening 117 (“emission aperture”). The emission aperture 117 is fluidly coupled to one or more channels 130, which are disposed within the stem 120 and are configured to enable fluid flow (e.g., an irrigant) from the vessel 105 to the outlet 125. In various embodiments, the deployment head 110 may be formed as a unitary component, where each of the base 115, stem 120, and outlet 125 are integrally formed. In other embodiments, the deployment head 110 may be formed as a collection of components, where each of the base 115, stem 120, and/or outlet 125 may be coupled (removably or permanently) to form the deployment head 110. For example, in various embodiments, the base 115, stem 120, and/or outlet 125 may be coupled via friction or interference fit, one or more threads, an adhesive, etc.

In various embodiments, the outlet 125 (and the opening 117) may include one or more nozzles to increase an exit velocity of the fluid. In other embodiments, the outlet 125 and opening 117 may include a plurality of channels to enable dispersal of the irrigation fluid within a wide area (i.e., within an area greater than a width of the outlet 125), in a specific direction (e.g., parallel to an axis of the stem 120 and/or outlet 125, at a predetermined angle relative to the outlet 125, etc.), and/or to change a velocity of the irrigant as it flows from the outlet 125 (e.g., reducing fluid velocity to minimize splashing unintended areas, and spreading matter from a wound site). In various embodiments, the deployment head 110 may alternatively or additionally include one or more apertures disposed within the stem 120 prior to the outlet 125, where the one or more apertures may enable irrigant to be expelled along a length of the stem 120. As described above, the vessel 105 may be structured to be substantially rigid, flexible, elastic, etc. In at least one embodiment, the vessel 105 may be a an elastic bottle that can be deformed by manual manipulation (e.g., squeezed) to expel a fluid through the deployment head 110, where the vessel 105 is configured to return to its original shape after pressure is released (e.g., by receiving air through the deployment head after manual manipulation is ceased). In another embodiment, the vessel 105 may be substantially rigid such that it does not deform under force (e.g., from manual manipulation), and may instead be configured to expel irrigant in an inverted positon or by the aid of a pressurized fluid.

FIGS. 2A and 2B show various connection mechanisms between the base 115 of the deployment head 110 and the opening 107 of the vessel. As shown in FIG. 2B, a trocar (i.e., spike) 145 may be used to facilitate coupling of the stem 120 to the vessel 100. In some embodiments, the stem 120 may include one or more alternate connectors in addition to or instead of a trocar. As shown in FIG. 2A, the base 115 may include a first portion 135, which is configured to engage with the opening 107 (e.g., via one or more threads, friction fit, interference fit, etc.). A second portion 140 of the base 115 may extend outwardly from the first portion 135 and may be configured to receive or otherwise engage with (e.g., via one or more threads, friction fit, interference fit, etc.) the stem 120. As shown in FIG. 2B, the base 115 may be configured to receive the trocar 145 through each of the second portion 140 and the first portion 135 such that the trocar 145 extends into the vessel 105 to facilitate fluid communication between the vessel 105 and the stem 120. The stem 120 may include a ridge, flange, or collar 150, which is configured to interface or engage with the second portion 140 of the base 115 to facilitate coupling of the stem 120 to the base 115. In some embodiments, the irrigant vessel system 100 may be configured such that the opening 107 of the vessel 105 includes the base 115 (i.e., the base 115 is separate from the deployment head 110), where the deployment head 110 is coupled to the vessel 105 by connecting the stem 120 to the base 115.

Although FIGS. 2A and 2B depict the base 115 and stem 120 coupled to the vessel 105, in various embodiments, the base 115 and stem 120 may be similarly coupled to a secondary source reservoir (e.g., and additional bag, bottle, jug, etc.) that is separate from the vessel 105. In various embodiments, the base 115 may include one or more seals (e.g., a hermetic seal, a tamper seal, etc.) 137 dispose along an axis perpendicular to the opening 107 of the vessel 105. In various embodiments, the at least one seal 137 may be disposed between the first portion 135 and the second portion 140 of the base 115. In some embodiments, the second portion 140 may have a smaller width or diameter as compared to the first portion 135. In various embodiments, the at least one seal 137 may enable a sterile solution to be stored in the vessel 105 prior to its use (i.e., prior to being expelled from the vessel 105 through the deployment head 110).

In some embodiments, the base 115 may not include a seal 137. In other embodiments, the base 115 may include a seal 137 disposed along an upper surface, such as within the second portion 140 (which may prevent particulate build up along a surface of the seal 137), or a lower surface, such as within the first portion 135 (which may enable the seal 137 to be broken upon coupling the base 115 to the opening 107 of the vessel 105). In some embodiments, the vessel 105 may itself include one or more integrated seals, which may evidence a first use of the vessel 105 to prevent undesired re-use (e.g., for a single use container or a container requiring a sterilization process prior to reuse). In some embodiments, the integrated seal may be unidirectional (e.g., a flutter valve), which may allow, for example, an egress of fluid from the vessel 105 but no ingress. In other embodiments, other seals, ports, etc. disclosed herein may make use of such valves.

As described above, FIG. 2B shows the trocar 145 interfacing with the vessel 105. The trocar 145 may be used to couple the deployment head 110 to the irrigating vessel 105 for operation. In other embodiments, the trocar 145 may be separate from the deployment head 110 and may be used to couple a secondary source reservoir to fill or re-fill the irrigating vessel, etc. The trocar 145 is shown piercing the seal 137 disposed within the base 115. In some embodiments, the seal 137 may be self-healing (e.g., a rubberized seal) and may maintain a seal after withdrawal of the trocar 145 from the base 115 and/or vessel 105. In various embodiments, the seal 137 may allow for the formulation of a fluid (e.g., an irrigant) within the container. In some embodiments, the seal 137 may be a tamper seal, which may enable visual identification of a previously used base 115, vessel 105, etc. In various embodiments, the trocar 145 (or a similar assembly) may be configured to attach to the vessel 105 and/or the base 115 via one or more threads, adhesives, and/or other various needleless connectors or mechanisms known in the art. In some embodiments, the trocar 145 may be sized to reach a bottom surface of the vessel 105 when coupled thereto. In some embodiments, the base 115 and/or vessel 105 is configured to limit ingress of the trocar 145 beyond the seal 137 (e.g., to minimize unusable fluid where the vessel 105 is intended to be inverted during operation).

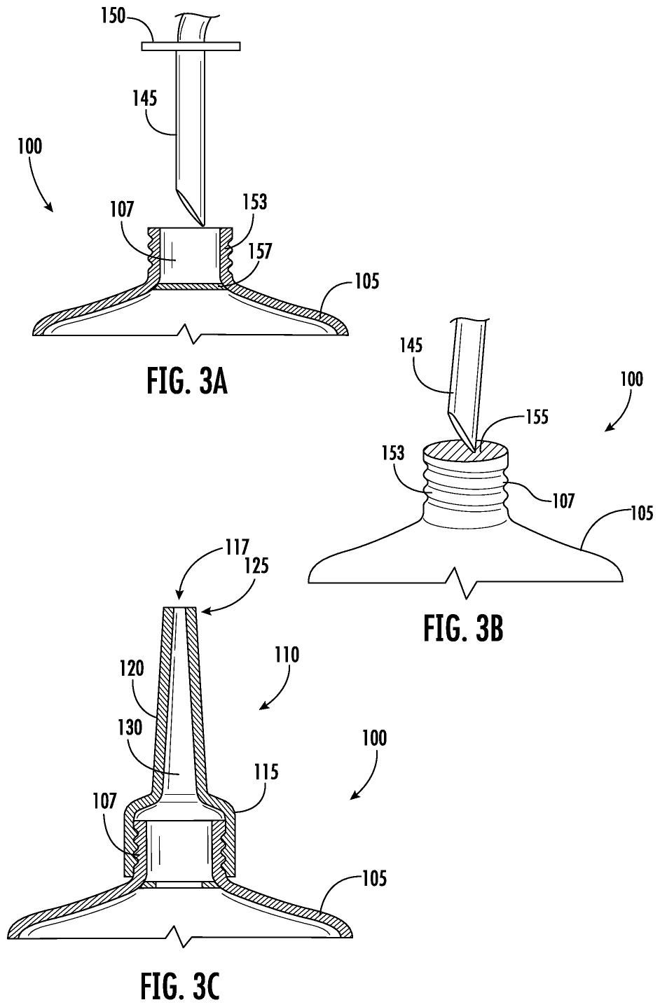

In some embodiments, the vessel 105 may include one or more features to facilitate deployment of irrigant, wound preparation, and/or debridement. FIGS. 3A and 3B show two additional configurations of the vessel 105 coupled to the trocar 145. FIG. 3A shows a cross section view of the vessel 105 near the opening 107, where the opening 107 includes a plurality of threads 153 to facilitate coupling of the trocar 145 (or other components) thereto. As shown, the opening 107 also includes at least one seal 157 disposed at a base of the threads 153 of the opening 107. In other embodiments, such as shown in FIG. 3B, the vessel 105 may be structured to include a seal 155 disposed at an upper surface of the opening. It should be noted that although FIGS. 3A and 3B show the vessel 105, in various embodiments, the irrigation system 100 may include one or more additional containers, which may be structurally similar or equivalent to the vessel 105 such that the system 100 includes two or more separate containers (e.g., which may be joined by the trocar 145 or a trocar assembly for the purpose refilling an irrigating vessel from an separate source). In some embodiments, at least one of the containers is sealed after being filled with an irrigant solution. In various embodiments, the irrigant solution may include at least one of saline, lactated ringers, sterile water, betadine, gentamicin, vancomycin, bacitracin, chlorhexidine, povidine iodine, hydrogen peroxide, chlorhexidine gluconate (CHG), acetic acid, sodium hypochlorite, or hypochlorous acid. In some embodiments, the containers are configured to formulate irrigants in situ, or may be filled with a preformulated shelf stable formulation.

In some embodiments, the deployment head 110 may be a unitary component having a tapered stem 120, which terminates at the opening 117. FIG. 3C shows the deployment head 110 coupled to the vessel 105, where the opening 107 of the vessel 105 includes a pierced seal, where the pierced seal may be breached by the deployment head 110, the trocar 145 (shown in FIG. 3A), or some other mechanism. As shown, the deployment head 110 may be configured such that the stem 120 has a generally tapered structure, decreasing in width as it extends away from the base 115. Although FIG. 3C shows the deployment head as having a primary axis that is generally aligned with a primary longitudinal axis of the vessel 105, in various embodiments, the deployment head 110 may extend in any direction relative to the vessel 105.

As described, the deployment head 110 may facilitate deployment of irrigation solution from the vessel 105 through the stem 120 (i.e., elongated member) having the inner channel 130, which leads to the opening 117 (i.e., aperture) through which the solution is dispersed. In various embodiments, the inner channel 130 may include one or more sub-channels, which each terminate at an aperture disposed at the opening 117. For example, in various embodiments, the channel 130 may include two or four sub-channels such that the opening correspondingly includes two or four apertures through which irrigating solution may be dispersed. In various embodiments, the stem 120 may be structured to have a length, inner (i.e., core) diameter, outer diameter, and/or angulation designed to most effectively and ergonomically facilitate irrigation and/or debridement. In at least one embodiment, the stem 120 may be tapered along its outer and/or inner diameter from the base 115 (i.e., adjacent to the vessel 105) to the outlet 125 (i.e., which includes the opening 117). Accordingly, the tapered stem 120 may control a resistance, velocity, and flow of the irrigation solution as it exits the vessel 105. Thus, in various embodiments, the dimensions of the stem 120 (and/or of the base 115 and outlet 125) can be modified to influence the pressure and stream characteristics of irrigation solution flowing from the vessel 105 even when one or more forces (e.g., due to manual manipulation) are applied to the vessel 105.

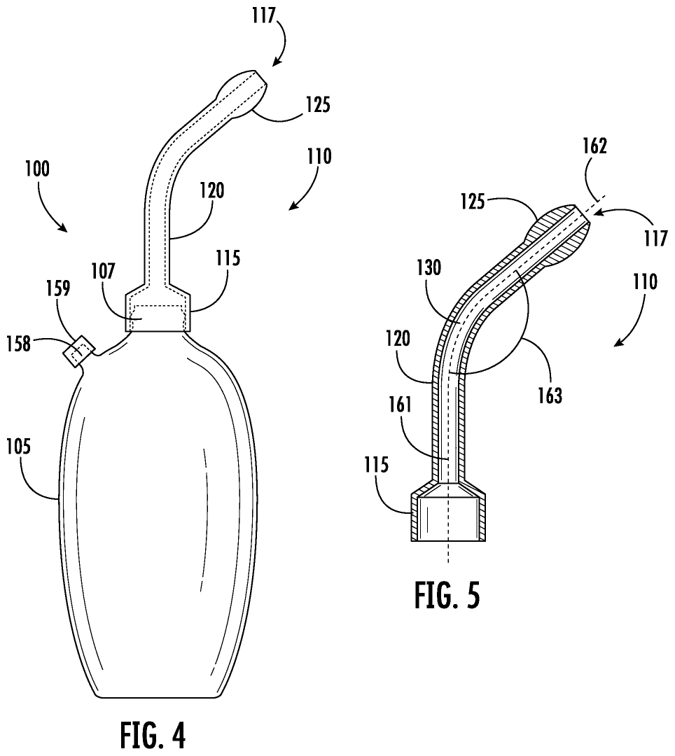

In various embodiments, the vessel 105 may include more than one opening. As shown in FIG. 4, the vessel 105 may include a second opening (e.g., receiving port) 158 of the vessel. As shown, the second opening 158 may include a barrier 159 (e.g., cap, cover, etc.), which is configured to cover the opening 158 during operation of the irrigation vessel system 100. In various embodiments, the barrier 159 may include one or more seals (e.g., similar or equivalent to the seals 137, 155, and/or 157) or interfaces (e.g., similar or equivalent to the threads 153). For example, the barrier 159 may be a self-healing seal, a threaded cap, etc. In some embodiments, the barrier 159 may a valve for the irrigating vessel 105. In such an embodiment, removal of the barrier 159 may allow air to enter the vessel 105 (i.e., after the vessel 105 has been squeezed), which may enable the vessel 105 to return to an undeformed shape without drawing air through the deployment head 110. Accordingly, the opening 158 and barrier 159 may, for example, prevent introduction of debris from a wound site or other site of interest into the vessel 105, which may clog one or more portions of the stem 120 (e.g., the opening 117, the channel 130, etc.) and/or damage a site of interest (e.g., by tearing tissues adhered to the deployment head 110 through suction).

In some embodiments, the second opening 158 may be closed during operation of the irrigation vessel system 100 to prevent particulate matter or other debris in the air from entering the vessel 105. A closed second opening 158 may also prevent unintended fluid discharge from the deployment head 110 (e.g., if an atmospheric pressure exceeds the pressure within the vessel 105). Accordingly, the second opening 158 may be opened selectively, e.g., as a valve, to control an amount and/or a rate of irrigant flow from the vessel 105. In various embodiments, a valve may also be disposed within the deployment head 110 itself, which may also control an amount and/or rate of irrigant flow. In various embodiments, the valve may include a push/pull interface, a twist control, and/or other related mechanism to facilitate control of irrigant flow.

FIG. 5 shows the deployment head 110, according to at least one embodiment. As shown, and as previously described, the deployment head 110 includes a stem (i.e., elongated spout or member) having at least one inner channel 130 (terminating in at least one opening 117), and length/core dimensions optimized to produce a specific quality, character, flow, and velocity of the irrigant flow from the vessel 105 during deployment. As shown in FIG. 5, the stem 120 may also be angled, bent, or rounded to allow ergonomic delivery of irrigant. As shown, the stem 120 may be aligned with a first axis 161 within a first region and aligned with a second axis 162 within a second region 162, where an angle 163 defined between the axes 161 and 162. In some embodiments, the angle 163 may be 10, 15, 30, 45, 75, or 90 degrees, or any other suitable angle for facilitating ergonomic delivery of irrigant. In various embodiments, the stem 120 may be formed of a flexible (i.e., elastic) material such that a user may adjust the stem 120 to have a specific angulation for providing irrigant to a site. An angled, elongated stem 120 would permit ergonomic use because the system 100 (including both the vessel 105 and the deployment head 110) could be positioned (e.g., as held by a user) at a variety of angles relative to a plane of the target (i.e., a wound, incision site, etc.) and facilitate effective irrigant delivery. Such a configuration also facilitates users' freedom of movement and positioning to access contours and recesses of complex or irregularly shaped structures and surfaces (i.e., of the wound, incision site, etc.) while also staying clear of the potential splash production.

In at least one embodiment, the deployment head 110 (e.g., via the stem 120) may be sufficiently flexible to be moved to a desired position, but sufficiently rigid to hold said desired position to enable a user to configure the direction and angle of the deployment head 110 during use. In various embodiments, the stem 120 may include one or more materials, such as a polymer or metal, which may plastically deform responsive to an applied force (e.g., manual manipulation at room temperature, with a tool at elevated temperature, etc.). In some embodiments, the stem 120 may be configured to elastically deform (e.g., to avoid damaging sensitive tissue) or to resist deformation (e.g., to enable vigorous debridement of a wound site).

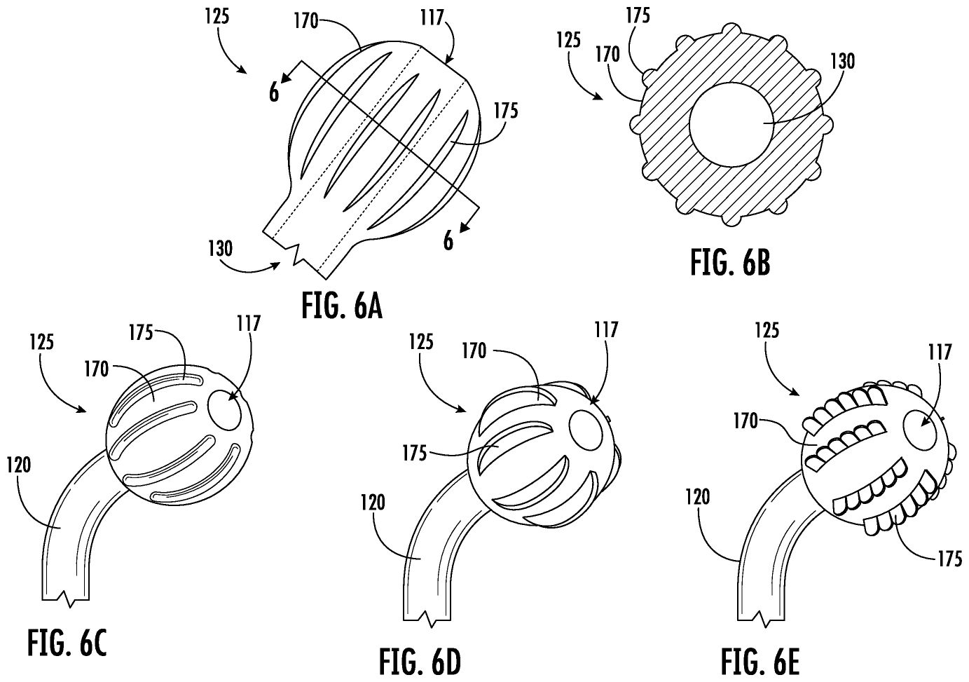

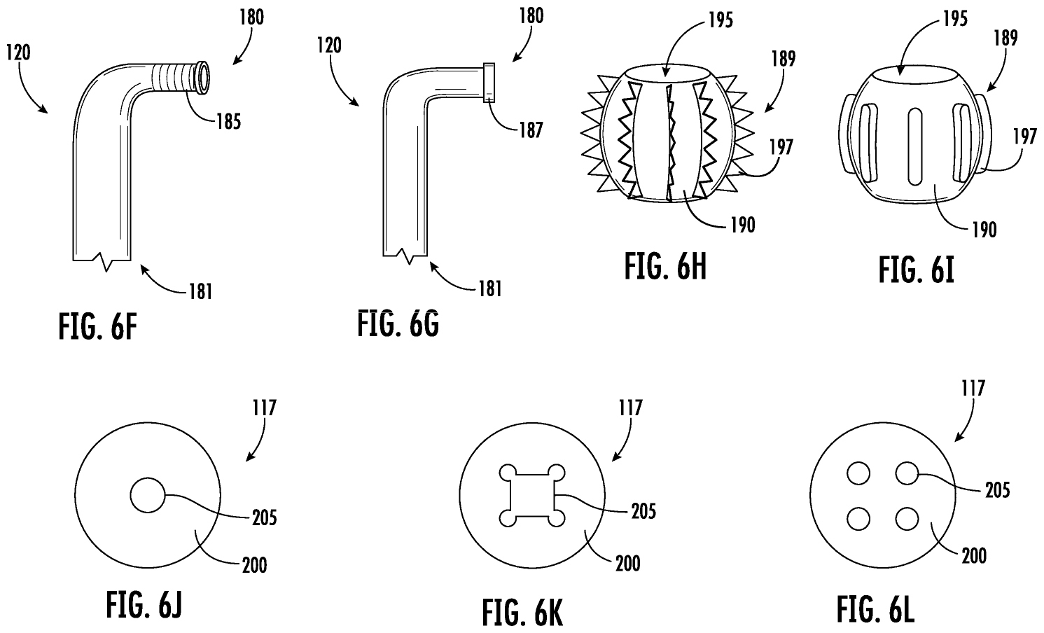

As described previously, the stem 120 of the deployment head 110 terminates at the outlet 125. In various embodiments, the outlet 125 may be structured to have a geometric design and dimensions to enable use a probe and/or mechanical debrider during irrigation. In some embodiments, such as shown in FIGS. 6A-6E, the outlet 125 is configured to have a generally bulbous shape. In some embodiments, the outlet 125 may have a smooth surface. Accordingly, the stem 120 and the outlet 125 may serve as a wand to facilitate probing in cavities or irregular contours (i.e., of the wound, incision, etc.) during irrigation. In various embodiments, stem 120 and the outlet 125 (“wand”) may be employed in a tactile fashion to mechanically loosen and release tissues, debris, foreign materials, etc. from a wound, incision, etc.

FIG. 6A shows the outlet 125, according to at least one embodiment. As illustrated, the outlet 125 includes a coarse outer surface 170, which has regularly spaced protrusions 175 disposed about the surface 170 of the bulbous outlet 125. As shown in FIG. 6A, the protrusions 175 may be structured as ridges which extend longitudinally along the outlet 125. The protrusions 175 may be circumferentially spaced about the outer surface 170. In some embodiments, at least one of a height, width, or length of the protrusions 175 may be set based on a desired amount of abrasiveness or mechanical engagement with the wound or incision. FIG. 6B shows a cross-sectional view of the outlet 125 taken along line 6-6 of FIG. 6A. As shown in FIG. 6B, the protrusions 175 are disposed about the outer surface 170 of the bulbous outlet 125. Although FIGS. 6A and 6B illustrate the protrusions 175 as elongated ridges, other embodiments of the outlet 125 may alternatively or additionally include various debriding features such as serrations, furrows, or other features configured to facilitate debridement. In other embodiments, such as shown in FIG. 6C, the protrusions 175 may be configured as grooves or furrows disposed about a circumference of the outlet 125. In other embodiments, such as shown in FIG. 6D, the protrusions 175 may be structured as fins extending outwardly from the outlet 125. In yet other embodiments, such as shown in FIG. 6E, the protrusions 175 may form spikes, bristles, or serrations extending from the outlet 125. It should be noted that although FIGS. 6A-6E show the protrusions as substantially aligned with a longitudinal axis of the stem 120 and outlet 125, in some embodiments, the protrusions 175 may be arranged to be substantially perpendicular to the stem 120. In yet other embodiments, the protrusions 175 may be irregularly spaced about the outlet 125 (e.g., the protrusions 175 may be concentrated within one region of the outlet 125 to selectively debride and irrigate a wound without undesired tissue abrasion). In various embodiments, the protrusions 175 may be substantially rigid. In other embodiments, the protrusions 175 may be flexible or otherwise deformable. In some embodiments, the deployment head 110 or portions thereof (e.g., the outlet 125) may include or be coupled to one or more ultrasonic components such that the deployment head 110 may facilitate debridement using ultrasound.

In various embodiments, the deployment head 110 may be modular in structure and may thus be configured to receive one or more different outlets 125 or portions thereof. In other embodiments, the irrigant vessel system 100 may include a plurality of deployment heads 110, which are each configured to removably couple to the irrigant vessel 105 to allow for the selection of an appropriate working surface or angle (e.g., a working surface of the outlet 125, an angle or length of the stem 120, etc.) for a particular use. For example, a relatively aggressive working surface (i.e., formed by the surface 170 and the protrusions 175) and contour design (i.e., formed by a length and angle of the stem 120) may be appropriate for a chronic open soft tissue wound, whereas a milder contour design would be preferred to debride and irrigate delicate tissues (e.g., dura) to minimize a risk of injury. As shown in FIGS. 6F and 6G, the stem 120 of the deployment head 110 may be configured to receive the outlet 125 at an end thereof. As shown, the stem 120 includes a first end 181, which is configured to couple to the base 115, and a second end 180, which is configured to couple to the outlet 125. The second end 180 may include threads 185 to facilitate threadably coupling the outlet 125 to the stem 120. In other embodiments, the second end 180 may include one or more ridges, rims, flanges, or other protruding features 187, which may facilitate an interference fit between the stem 120 and the outlet 125. For example, in some embodiments, the outlet 125 may be configured to snap or thread onto the second end 180 of the stem to allow switching between various outlets 125, which may each have a different working surface (e.g., one outlet 125 may have ridges, another outlet 125 may include bristles, another outlet 125 may include fins, etc., where each outlet 125 may have a different use application or use profile). In yet other embodiments, the stem 120 may be configured to couple to the outlet 125 via friction fit. In some embodiments, the outlet 125 itself may include one or more modular components. Accordingly, in addition to or instead of replacing the outlet 125 entirely, only portion (e.g., a tip of the outlet 125) may be replaced to change a working surface of the outlet 125. For example, as shown in FIGS. 6H and 6I, a sleeve 189 may be configured to couple to the outlet 125 (e.g., by receiving the outlet within an inner bore 195), where the sleeve 189 may include an outer surface 190 having a plurality of protruding members 197 extending therefrom. In some embodiments, the sleeve 189 may be configured to couple to the outlet 125 via snap fit, friction fit, interference fit, etc. As shown in FIG. 6H, the one or more protruding members 197 may be formed as spikes or serrations disposed in substantially linear sections about a circumference of the outer surface 190. In other embodiments, such as shown in FIG. 6I, the one or more protruding members 197 may be formed as ridges disposed about the circumference of the outer surface 190. Accordingly, during use of the system 100, different working surfaces may be replaceably switched at the outlet 125 of the deployment head 110 by replacing the outlet 125 entirely (e.g., via coupling different outlets 125 to the stem 120) or by replacing a portion of the outlet 125 (e.g., via switching sleeves 189). In various embodiments, the outlet 125 or a portion thereof may be replaced or switched to facilitate different debridement steps and/or to otherwise allow for different uses of the deployment head 110. For example, various outlets 125 may have working surfaces with different levels of abrasion or debridement capabilities, or some outlets 125 may be better suited to irrigate or debride different wound types or areas.

As described previously, the channel 130 may include one or more sub channels, which terminate at the opening 117. As shown in FIGS. 6J-6L, the opening 117 may be configured to include one or more apertures 205, which allow outflow of irrigant from the vessel 105 through the channel 130. In various embodiments, the opening 117 may be formed by a single aperture 205 disposed within a terminal surface 200 of the outlet 125, such as shown in FIG. 6J. In other embodiments, the aperture 205 may have a variable shape such that a width or diameter of the aperture 205 varies, such as shown in FIG. 6K. In yet other embodiments, the opening 117 may be formed by a plurality of apertures 205 (e.g., 2, 4, 6, 7, 12, etc.) disposed within the terminal surface 200 of the outlet 117, such as such in FIG. 6L.

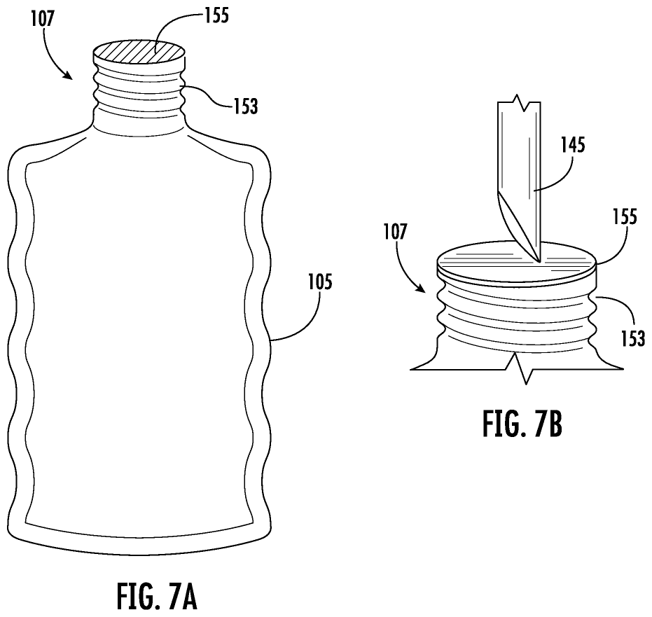

In various embodiments, the vessel 105 may be formed from a flexible and readily deformable material. For example, as shown in FIG. 7A, the vessel 105 may be non-rigid body (i.e., a bag). As shown, the vessel 105 may include at least one seal (e.g., similar or equivalent to the seal 155) disposed at the opening 107, where the at least one seal may maintain sterility of a fluid (e.g., sterile irrigant) in the vessel 105. In various embodiments, the opening 107 of the vessel 105 may include one or more interfaces, such as the threads 153 to facilitate coupling of the deployment head 110 thereto.

As described above, in some embodiments, the irrigation vessel system 100 may include multiple vessels 105 or containers. Accordingly, in some embodiments, at least one vessel 105 may be a secondary reservoir or irrigant source, which can be used to fill or refill a primary irrigant vessel 105. In various embodiments, the secondary vessel 105 may include a hanging element (e.g., a hook, a hole, a clip, etc.), which may be used to invert and/or raise the secondary vessel 105 to aid in transferring a fluid (e.g., irrigant) from the secondary vessel 105 to the primary irrigant vessel 105, to a deployment head 110, etc. As shown in FIG. 7B, the seal 155 of the vessel 105 may be breeched by a trocar (similar or equivalent to the trocar 145) or other connecting element which may be coupled to the opening 107 (e.g., by a base 115 of another vessel 105, medical tubing, etc.). In at least one embodiment, the vessel 105 may be coupled to a deployment head 110 through intermediate tubing. As discussed above, the deployment head 110 may include one or more valves to control flow of irrigant out of the vessel 105. Such a configuration may allow for greater dexterity when using the irrigation vessel system 100 for irrigating a site, as a user may simply manipulate the deployment head 110 (which may be connected to the vessel 105 via tubing) rather than holding the irrigant vessel 105 itself (or another irrigant reservoir), which may be bulky or heavy.

In various embodiments where the irrigation vessel system 100 includes more than one vessel 105, a first vessel 105 may be configured to attach to a second vessel 105 (e.g., directly or through an intermediate connection such as tubing). For example, a first vessel 105 may be coupled to a second vessel 105 via a second opening 158 (i.e., a receiving port) of the second vessel 105, where the second vessel 105 is further coupled to a deployment head 110 (i.e., via the opening 107 of the second vessel 105). Accordingly, the second vessel 105 may provide additional irrigant (i.e., in addition to the irrigant contained in the first vessel 105) to allow continuous irrigation (e.g., by a medical professional or other user) of a site (e.g., wound, incision, etc.). In some embodiments, an intermediate connector configured to connect between the first vessel 105 and the second vessel 105 may enable the second vessel 105 to be replaced during use, which may allow for providing a limitless source of irrigant that may be used without interruption to promote wound care and better patient outcomes.

Another aspect of the present disclosure relates to lightweight, practical, and effective methods and devices to perform medical and/or surgical irrigation in a wide range of clinical care environments. In at least one embodiment, the irrigation device includes a compressible container designed to accept an irrigation solution under sterile conditions from a secondary source (e.g., an intravenous solution bag), where the solution may reconstitute or dilute an active agent such as an antimicrobial, which is either pre-set in the container or added to the container in addition to a diluent solution. When the irrigation device is coupled to the secondary source, the irrigation device may deliver irrigant to a medical/surgical site. In some embodiments, the irrigation device may directly delivery irrigant. In other embodiments, the irrigation device may deliver irrigant via a delivery accessory. In yet other embodiments, the irrigation device may deliver irrigant responsive to an applied pressure.

Referring generally to the figures, a device to accept and deploy an irrigating solution to a desired site (i.e. anatomic area, cavity, orifice, or wound) is shown. In some embodiments, the irrigation device is a fillable, collapsible, or compressible container made of a biocompatible material. In some embodiments, the irrigation device may be a bottle, a bag, or other vessel capable of accepting, holding, and/or deploying liquid contents. In various embodiments, the irrigation device may be sterilizable (e.g., via an autoclave, chemical treatment, etc.).

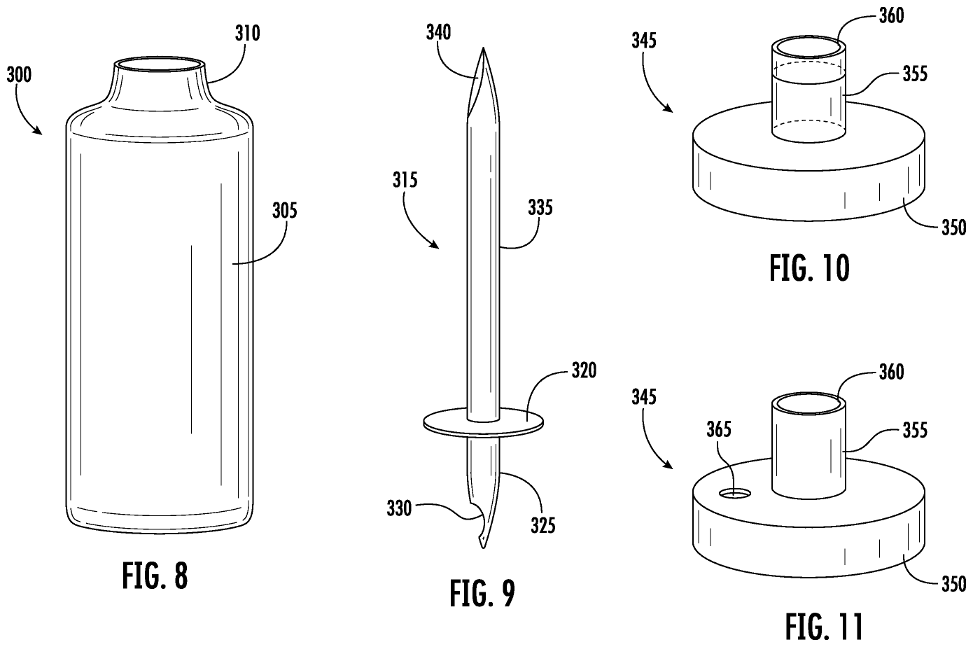

FIG. 8 shows an irrigation device 300, according to at least one embodiment. In some embodiments, the device 300 may be similar or equivalent to the vessel 105. As shown in FIG. 8, the device 300 may include an enclosed body 305 having at least one opening 310. The body 305 may be made of one or more flexible (i.e., elastic) materials configured to elastically deform in response to an applied load. In other embodiments, the body 305 may be substantially rigid, having only selective portions thereof that elastically deform in response to an applied load (e.g., squeezing by a user). In various embodiments, the device 300 may include or be configured to couple to one or more components, which may facilitate fluid transfer into or out of the device 300. In some embodiments, the one or more components may include an access site, such as a cap, fill port, or another apparatus or means for facilitating transfer of irrigant (or irrigant solution) from a secondary source (e.g., a reservoir) to the device 300, and/or to facilitate deployment of irrigant (or irrigant solution) from the device 300 to a target site.

In various embodiments, the device 300 may be filled with a solution or diluent for medical and/or surgical irrigation. Such solutions or diluents may include, but are not limited to, saline, lactated ringers, sterile water, and non-sterile water. In some instances, the solution may be specifically prepared for a particular purpose and may include one or more active agents, such as a chemical disinfectant, antibacterial agent, and/or antiviral agent. In various embodiments, when filled with an irrigant (i.e., a solution or diluent) and coupled with a mechanism to facilitate delivery of the irrigant to a specific site, the device 300 may be compressed to deploy irrigant to the site at a pressure that corresponds to a degree of compressive force applied to the device 300 (i.e., via squeezing or other manipulation).

As described above, the device 300 may include one or more openings 310 (i.e., access sites) through which to accept an irrigant solution and/or to deploy it for use. Prior to filling, the device 300 may be a fully closed and sterile system, where the one or more openings 310 are sealed such that the device 300 is both watertight and airtight. In at least one embodiment, the device 300 may be collapsed, compressed, and/or evacuated by negative pressure prior to filling with an irrigant solution. In such embodiments, the device 300 may be configured to be compact and lightweight. In other embodiments, the empty device 300 may have a physical form, which is similar or identical to its form in a filled state.

As described above, the one or more openings 310 are configured to accept delivery of a desired irrigant/diluent solution into the device 300 and/or to facilitate deployment of the irrigant/diluent solution to a specific site. In various embodiments, the one or more openings 310 may include or be couplable to a cap, stopper, threaded or non-threaded top, integrated port, and/or a tubing connector. In at least one embodiment, the one or more openings 310 are configured to facilitate filling of the device 300 under sterile conditions such that the device 300 may be maintained in a sterile state. In various embodiments, the one or more openings 310 may include one or more mechanisms (e.g., a valve) to release pressure, which may rise within the device 300 during the process of filling.

In general, solutions to be used for medical or surgical irrigation are preferably sterile. Accordingly, the container device 300 may be filled in a transfer process from a secondary source of sterile solution. In some embodiments, an accessory device may be coupled to the container device 300 (e.g., via the one or more openings 310) to facilitate transfer of irrigant solution from a secondary source to the device 300 in a manner that maintains sterility of the irrigant solution. Thus, the accessory device and process should be a closed system.

In various embodiments, the accessory device may include a decanter 315, such as shown in FIG. 9. The decanter 315 may include a first portion 325 and a second portion 335, which together form a hollow stem. The first portion 325 may be configured for insertion into the opening 310, where the first portion 315 may itself include one or more openings 330 disposed at a terminal end thereof. The one or more openings 330 may enable irrigant from within the device 300 to enter the decanter 315 to be transferred out of the decanter 315. As shown in FIG. 9, the one or more openings 330 may be angled, scalloped, or otherwise contoured to facilitate entry of irrigant. In some embodiments, the terminal end of the first portion 325 may be sufficiently sharp such the chat it may be used to pierce a seal disposed at the opening 310. The second portion 335 may also include one or more openings 340 disposed at a terminal end thereof, where the one or more openings 340 may facilitate egress of irrigant. In various embodiments, the one or more openings 340 may be angled, scalloped, or otherwise contoured to facilitate pouring or other dispersal of the irrigant from within the device 300. As shown, the decanter 315 may also include a flange 320 disposed between the first and second portions 325, 335, where the flange 320 is configured to limit an amount of insertion of the decanter 315 into the opening 310. In some embodiments, the flange 320 may be configured to couple to at least a portion of the opening 310 to secure the decanter 315 to the device 300.

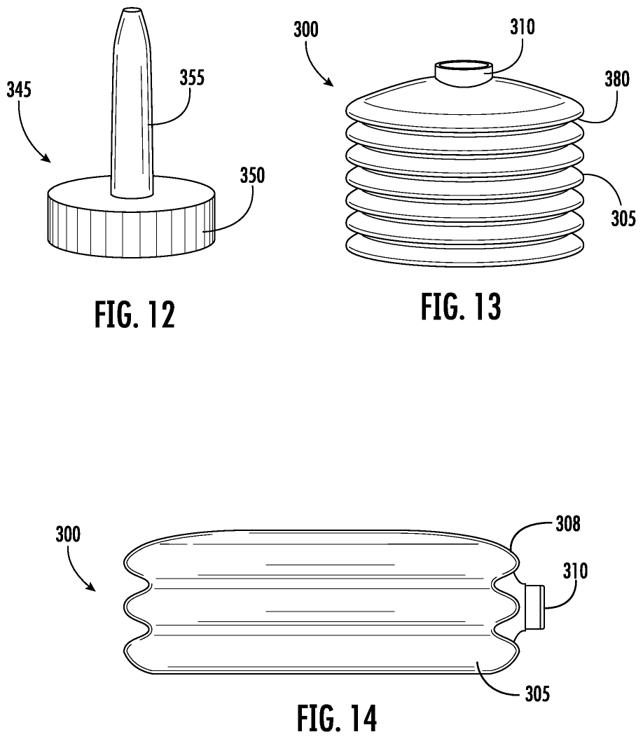

In other embodiments, the accessory device may include a cap 345, such as shown in FIGS. 10 and 11. As illustrated the cap 345 may include a base portion 350, which is configured to couple to the one or more openings 310 of the device 300. In some embodiments, the base portion 350 may include a plurality of threads, which are configured to engage with corresponding threads of the opening 310. In other embodiments, the base portion 350 may be configured to couple to the opening 310 via a friction or interference fit. As shown, the base portion 350 may be coupled to or integrally formed with a spout portion 355, which extends away from the base portion 350. In various embodiments, the spout portion 355 may have a smaller width or diameter as compared to the base portion 350. In various embodiments, the spout portion 355 may include at least one seal 360 disposed therein, where the at least one seal 360 is configured to prevent premature egress of irrigant from within the device 300 when the cap 345 is coupled to the opening 310. In some embodiments, the at least one seal 360 may be a self-healing seal, a frangible seal, a hermetic seal, or any other suitable seal type known in the art. In some embodiments, the seal 360 may be configured to be pierced by a separate piercing member (e.g., spike, needle, trocar, an end of the decanter 315, etc.) to facilitate egress of the irrigant from within the device 300. In some embodiments, such as shown in FIG. 11, the cap 345 may include a vent or valve 365 (“exhaust port”) disposed within the base portion 350 to facilitate pressure rebalance during filling or emptying of the device 300. In some embodiments, the valve 365 may be a one-way or a two-way valve. In some embodiments, the cap 345 may be configured for use during filling of the device 300 and/or during deployment of irrigant from within the device 300. Accordingly, in some embodiments, the cap 345 may be configured such that the spout portion 355 may be elongated to facilitate ease of dispersing irrigant to a specific site (e.g., wound, incision, etc.). As shown in FIG. 12, the spout portion 355 may be substantially cylindrical and having a tapered shape to enable control of an amount and/or rate of flow of irrigant.

In some embodiments, the device 300 may be structured to facilitate ease of irrigant dispersal. As described above, in various embodiments, the device 300 may be deformable or collapsible. For example, as shown in FIGS. 13 and 14, the device 300 may be structured to include a plurality of accordion folds 380 such that the device 300 may readily collapse in response to an applied compressive load, thereby forcing irrigant from the device 300. In some embodiments, the folds 380 may be aligned such that the device 300 collapses in a direction that is parallel to a longitudinal axis of the device 300 (i.e., as shown in FIG. 13). In other embodiments, the folds 380 may be aligned such that the device 300 collapses in a direction that is perpendicular to a longitudinal axis of the device 300 (i.e., as shown in FIG. 14). In various embodiments, the device 300 may be configured to readily inflate or expand in response to introduction of irrigant (i.e., during filling of the device 300). In various embodiments, multiple devices 300 may be used to transfer irrigant from a first device 300 to a second device 300 (e.g., a secondary source of sterile solution or irrigant), and/or from a first device 300 to a specific site (e.g., wound, incision, etc.).

In at least one embodiment, the secondary source of sterile solution may be a common intravenous solution bag (IV bag). Typically, an IV bag is accessed via a port site, which is sealed to the environment by means of a rubberized membrane within the port. To access an IV bag, a sharp, tubular member (i.e., a spike) may be used to penetrate the membrane of the port, thereby allowing egress of solution from within the IV bag via a further member (i.e., tubing, decanter, etc.) that is coupled to the spike. In various embodiments, a transfer device for facilitating solution transfer between the device 300 and a secondary solution source (e.g., IV bag) and/or additional device 300 may include a trocar or traditional IV bag spike. In other embodiments, alternative means for accessing the IV bag to transfer irrigant solution to the container device 300 may be used. In yet other embodiments, the secondary source of solution for filling the container device 300 may come from a type of sterile solution container that is different from the container device 300.

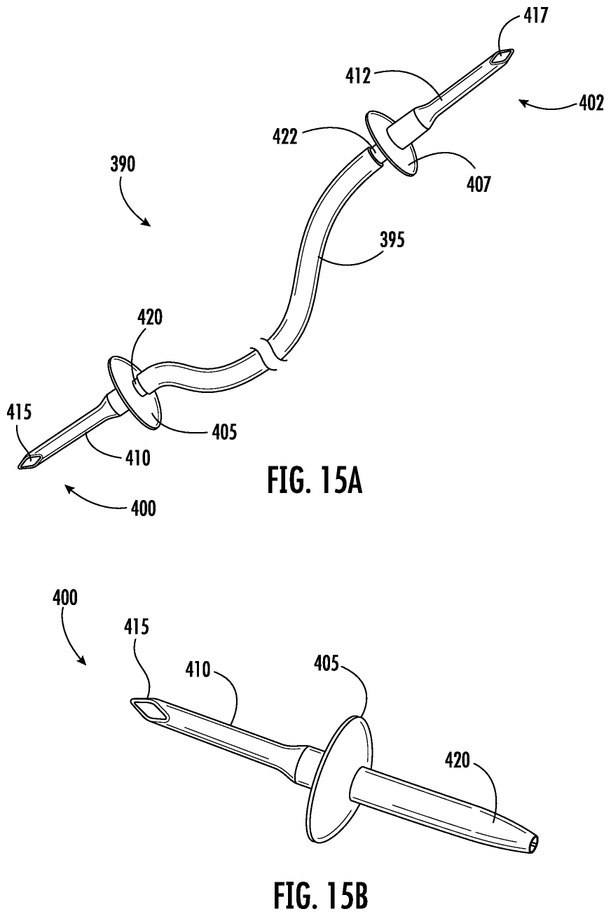

FIG. 15A shows a transfer device 390 for facilitating transfer of solution to or from the device 300. As shown, the transfer device 390 includes tubing 395 that extends between a first end 400 and a second end 402. At least one of the first end 400 or the second end 402 includes a trocar or other piercing member. As shown in FIGS. 15A and 15B, each of the first end 400 and second end 402 respectively include a trocar or piercing member 415, 417 (e.g., a common IV bag spike), which each include an elongated body 410, 412. The piercing members 415, 417 may respectively couple to or be integrally formed with a cap or flange 405, 407. Each of the caps 405, 407 may be configured to couple to an end of the tubing 395 such that the first end 400 is fluidly coupled to the second end 402. As shown, a receiving portion 420 of the cap 405 may extend outwardly from the cap 405 to receive an end of the tubing 395. Similarly, a receiving portion 422 of the cap 407 may extend outwardly from the cap 407 to receive another end of the tubing 395. In various embodiments, the tubing 395 may be or include rubber, silastic, plastic, and/or another flexible, biocompatible material.

In various embodiments, the device 300 may include a cap (e.g., similar or equivalent to the cap 345). The cap may be integrated with or separately coupled to the device 300 (e.g., via threading). In some embodiments, the cap itself may have an access port identical or similar to an IV bag access port. Accordingly, in some embodiments, a piercing member (e.g., the piercing member 415, or 417) of the transfer device 390 may be introduced to a secondary source (e.g., IV bag) via the access port and a membrane seal. Solution from the secondary source may then be transferred via this closed system to fill the device container 300. In some embodiments, solution transfer may be performed using alternative means to access the device container 300, such as via various cap designs, a luer lock, or other means of coupling. In yet other embodiments, the device container 300 may be filled in a non-closed system, where an access opening (e.g., the opening 310) may be used to transfer solution using an open pour, funnel, or similar technique.

As described above, the device container 300 may be filled with a solution to be used as an irrigant. In various embodiments, the irrigant may include, but is not limited to saline, lactated ringers, sterile water, non-sterile water, etc. In some embodiments, a compounded solution may be prepared for a specific use application. Such a compounded solution may include an active agent, such as a chemical disinfectant, antibacterial agent, and/or antiviral agent. In at least one embodiment, the device 300 may be filled with a pre-prepared compounded solution. In various embodiments, the device 300 in an empty state may be prepared by instillation of an active agent (i.e. antimicrobial, antiviral, etc.) to the device 300, where the active agent is in a solid, liquid, or gaseous form and is soluble upon introduction of diluent to the device 300. In some embodiments, the diluent may be introduced to the device 300 using a transfer process (i.e., using the transfer device 390) as previously described.

In various embodiments, a concentration of the irrigant solution is influenced by the mass of the solute agent and the volume of the diluent solvent. In some embodiments, a homogeneous or non-homogeneous solution may be prepared. In some embodiments, a desired concentration of any active agent can be pre-determined by altering either the solute mass held in the unfilled device 300, or the volume of diluent added to the device 300. In at least one embodiment, an optimal concentration and volume for the purpose of wound irrigation may be pre-determined, and a fill capacity of the device 300 and the mass of the added solute may be designed to meet specific needs. For example, a number of fill capacities and container sizes of the container 300 may be selected to match and/or accommodate volumes of the most common IV bag sizes: 100 ml, 250 ml, 500 ml, and 1000 ml. In various embodiments, desired concentration(s) of irrigants containing an active agent could be achieved in each of these volumes, all of which may have utility in various use applications.

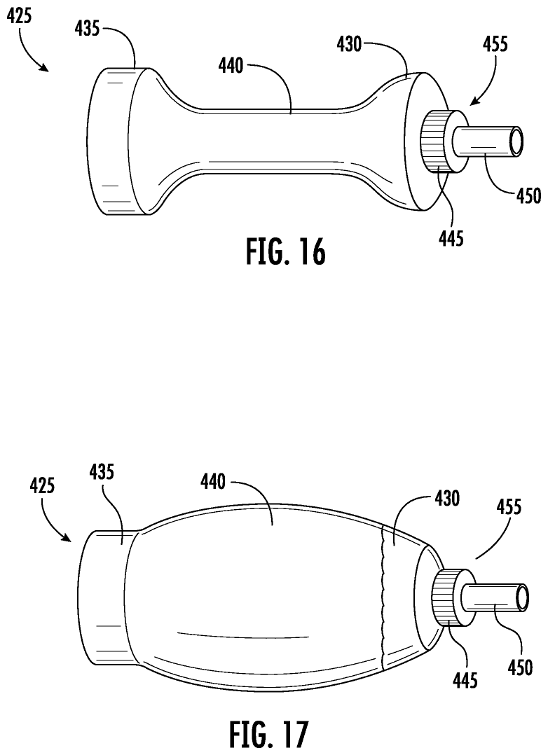

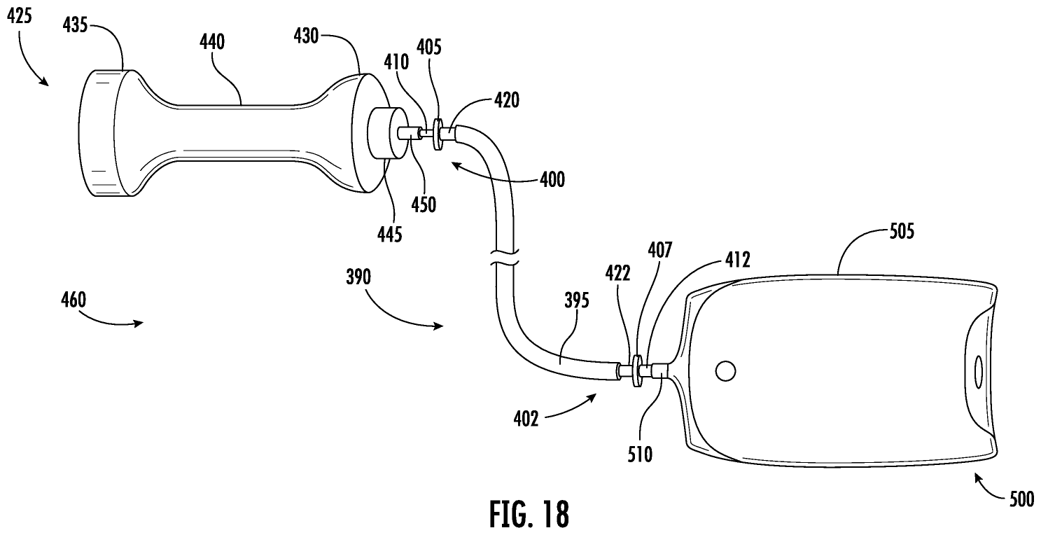

As described throughout the present disclosure containers for containing an irrigant solution may have a variety of shapes and sizes. FIGS. 16 and 17 show side views of a container device 425, which may be similar or equivalent to the device 300. As shown in FIG. 16, the device 425 may include a body 430 having one or more substantially rigid regions 435 and one or more substantially elastic regions 440. In some embodiments, the elastic regions 440 may have a smaller width or diameter than the rigid regions 435 when the device 425 is in an empty state. As shown, the device 425 may include one or more openings 455, which may be integrally formed with or configured to couple to a cap 445 (e.g., similar or equivalent to the cap 345), which has a spout portion 450 (e.g., similar or equivalent to the spout portion 355). As shown in FIG. 17, the device 425 may expand (i.e., within the flexible region 440) to increase in volume when being filled with solution. FIG. 18 shows an irrigation system 460, according to at least one embodiment. The irrigation system 460 includes the device 425, which may be fluidly coupled to a secondary source 500 via the transfer device 390. As shown, the device 425 may be coupled to the first end 400 of the transfer device 390. The first end 400 may be coupled to the device 425 via the piercing member 415 piercing a seal of the spout portion 450 (or otherwise extending into the spout portion 450). The second end 402 may be coupled to the secondary source 500, via the piercing member 417 piercing a seal disposed within an opening (e.g., access port) 510 of the secondary source 500. In various embodiments, the secondary source 500 may be an IV bag. In other embodiments, the secondary source 500 may be another container or device configured to contain an irrigant solution, active agent, or diluent. In some embodiments, the secondary source 500 may be similar or equivalent to the device 300 and/or 425. The secondary source 500 may include a body 505, which has an internal volume and contains the irrigant solution, active agent, or diluent. In various embodiments, the body 505 of the secondary source 500 may be flexible (i.e., elastic, compressible, deformable, etc.) or rigid. In some embodiments, the system 460 may be configured such that at least one of the device 425 or the secondary source 500 may be hung or otherwise elevated such that fluid flow into or out of the device 425 and/or secondary source 500 is driven by pressure due to gravity.

In various embodiments, when the secondary source 500 has been prepared and a desired irrigant solution has been transferred to from the secondary source 500 to the device 425, irrigant from the device 425 may be delivered to the wound for treatment. In various embodiments, the device 425 (or device 300) may include one or more dispersal means to facilitate irrigation. In at least one embodiment, the device 425 (or the device 300) may include an integrated delivery or deployment head (e.g., separate from or integrated with the spout portion 450), where irrigant solution may be ejected through the delivery head upon compression of the device 425. The delivery head (e.g., similar or equivalent to the deployment head 110) may permit irrigation in a plurality of fluid streams based on a corresponding plurality of apertures (“ejection ports”) incorporated in the deploy head. In some embodiments, the delivery head may be similar or equivalent to the decanter 315, the cap 345, or the deployment head 110. In various embodiments, flow and velocity of the irrigant stream(s) exiting the device 425 may be influenced by the design of the delivery head and ports, as well as by a pressure (i.e., compression) applied to the device 425 in the course of device activation and use.

In some embodiments, the opening 445 (i.e., the fill access site used to facilitate transfer of a diluent or irrigant solution to the device 425) may be configured to also couple to the delivery head, such that irrigant may be deployed in a similar manner as described above. In such embodiments, the delivery head may include one or more piercing members (e.g., trocar, IV bag spike) to access the device 425 in a similar fashion as the transfer device 390. In yet other embodiments, the device 425 may include more than one opening such that the delivery head may couple to the device 425 at an opening other than the opening 445. For example, the delivery head may be configured to couple to a threaded cap, which is affixed to a secondary opening of the device 425.

As utilized herein with respect to numerical ranges, the terms “approximately,” “about,” “substantially,” and similar terms generally mean +/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data that cause a general-purpose computer, special purpose computer, or special purpose processing machines to perform a certain function or group of functions.

Although the figures and description may illustrate a specific order of method steps, the order of such steps may differ from what is depicted and described, unless specified differently above. In addition, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A system comprising:
an irrigation vessel comprising at least one opening and containing an irrigant solution; and
a deployment head configured to couple to the at least one opening and to facilitate egress of the irrigant solution from within the irrigation vessel;
wherein:
the deployment head, comprises:
a base configured to couple to the at least one opening;
a stem coupled to the base at a first end and comprising an inner channel in fluid communication with the irrigation vessel and configured to allow flow of the irrigant solution therethrough;
an outlet coupled to the base at a second end, the outlet having at least one aperture disposed therein, wherein the at least one aperture is fluidly coupled to the inner channel, and the at least one aperture is configured to expel the irrigant solution from the vessel; and
the outlet comprises a working surface configured to debride a wound site.

2. The system of claim 1, wherein the working surface is defined by an outer surface of the outlet, the outer surface comprising a plurality of protruding members extending from the outer surface, wherein each of the protruding members are configured to mechanically debride at least a portion of the wound site.

3. The system of claim 2, wherein the outlet is bulbous, and wherein the plurality of protruding members are disposed about a circumference of the outlet.

4. The system of claim 3, wherein the plurality of protruding members is arranged in substantially linear sections.

5. The system of claim 3, wherein the plurality of protruding members include ridges.

6. The system of claim 3, wherein the plurality of protruding members include furrows.

7. The system of claim 3, wherein the plurality of protruding members include spikes.

8. The system of claim 1, wherein the at least one opening of the irrigation vessel includes a first opening and a second opening.

9. The system of claim 9, further comprising a second container, the second container configured to contain a fluid, wherein the second container is further configured to couple to at least one of the first opening or the second opening.

10. The system of claim 9, wherein the deployment head is configured to couple to the first opening and the second container is configured to couple to the second opening.

11. The system of claim 1, wherein the irrigation vessel is configured to be removably coupled to the deployment head.

12. The system of claim 1, wherein the irrigant solution comprises chlorhexidine gluconate (CHG), saline, or a mixture thereof.

13. The system of claim 1, wherein the at least one opening of the irrigation vessel comprises at least one seal.

14. The system of claim 13, wherein the at least one seal is a hermetic seal.

15. A method of irrigating and debriding a wound site, the method comprising:
filling a vessel with an irrigant solution, the vessel comprising at least one opening;
coupling a deployment head to the at least one opening of the vessel; and
applying a force to the vessel, wherein applying the force to the vessel causes egress of the irrigant solution from the vessel through the deployment head;
wherein:
the deployment head comprises:
a base configured to couple to the at least one opening;
a stem fluidly coupled to the base, the stem having an inner channel that is in fluid communication with the irrigation vessel;
an outlet coupled to the base, the outlet having at least one aperture disposed therein, wherein the at least one aperture is fluidly coupled to the inner channel; and
the outlet comprises a working surface configured to debride a wound site.

16. The method of claim 15, wherein filling the vessel comprises coupling a second container to the at least one opening.

17. The method of claim 16, wherein coupling the second container to the at least one opening comprises inserting a trocar through a seal of the at least one opening.

18. The method of claim 15, further comprising adjusting an angle of the stem.

19. The method of claim 15, further comprising debriding the wound site by mechanically loosening at least one of tissue or debris from the wound site via one or more protrusions disposed on the outlet.

20. The method of claim 15, wherein the deployment head further comprises tubing extending between the base and the stem.
